(19) United States
(12) Patent Application Publication
(10) Pub. No.: US 2025/0114231 A1
(43) Pub. Date: Apr. 10, 2025

(54) Urinary Catheter System

(71) Applicant: C. R. Bard, Inc., Franklin Lakes, NJ (US)

(72) Inventors: Ronald N. Legaspi, Alpharetta, GA (US); Pooja Bhuvanesh Kulkarni, Pune (IN); Vinayak Kulkarni, Bangalore (IN); Jain Jose, Thrissur (IN)

(21) Appl. No.: 18/694,911

(22) PCT Filed: Sep. 28, 2022
(86) PCT No.: PCT/US22/45084

§ 371 Date: Mar. 22, 2024

Related U.S. Application Data
(60) Provisional application No. 63/250,010, filed on Sep. 29, 2021.

(51) Int. Cl. A61F 5/44; A61M 25/00

(52) U.S. Cl. CPC A61F 5/4408; A61F 5/4405; A61F 5/4407; A61M 25/002

(57) ABSTRACT

Disclosed herein is a urinary catheter system including a urinary catheter, a drainage tube connecting the urinary catheter to a urine collection bag. The urine collection bag is configured to transition between a storage state and a usage state. The urine collection bag includes an intake port coupled to a first opening, an outlet, where the outlet is configured to transition from a closed state to an opened state to allow the volume of fluid to be displaced from the urine collection bag. A storage bag detachably coupled to the urine collection bag, the storage bag having a storage bag opening and configured to receive the male urinary catheter, the drainage tube, and the urine collection bag in the storage state.

### PRIORITY

This application claims the benefit of priority to U.S. Provisional Application No. 63/250,010, filed Sep. 29, 2021, which is incorporated by reference in its entirety into this application.

### BACKGROUND

Urinary incontinence causes a person to lose control over their bladder resulting in loss of large or small quantities of urine. This can be caused due to multiple reasons. A paraplegic or tetraplegic user loses the control of their bladder and they need to empty their bladder using urinary catheters. These can be indwelling (the catheter is placed for a longer duration inside the bladder and attached to a urine bag to collect the urine) or they can be intermittent (the catheter is periodically inserted into the bladder and the urine is drained from it either directly into appropriate drainage receptacle or into a collection bag). Intermittent catheters are preferred in the cases where the user can self-catheterize or wishes to not be seen with a urine bag. The intermittent catheters allow the user a freedom of mobility.

In some instances, the user may drain his bladder directly into a drainage receptacle, such as a toilet or urinal. In other instances, a drainage receptacle may not be readily accessible. In such instances, the user may connect a bag to the catheter to drain the urine into the bag. In some instances, connecting the bag to the catheter may be inconvenient and may introduce an infection risk to the user. In some instances, a bag disconnected from the catheter may not be resealable, and as such, urine may spill from the bag.

### SUMMARY

Disclosed herein is a urinary catheter system that, according to some embodiments, includes a urinary catheter and urine collection bag, where the bag defines a top end and a bottom end, and where the urine collection bag coupled with the urinary catheter via a hub at the top end. The urine collection bag further includes (i) an inlet opening coupled with the hub, where the inlet opening is in fluid communication with a lumen of the urinary catheter via a lumen of the hub; (ii) an outlet configured to transition from a closed state to an opened state to allow urine to be drained from the urine collection bag; and (iii) a storage bag separably attached to the urine collection bag, where the storage bag is configured to receive therein the urinary catheter and the urine collection bag in a storage state via an opening of the storage bag.

In some embodiments of the system, the urinary catheter is a male urinary catheter.

In some embodiments, the system further includes a catheter cap having a pull ring, where the catheter cap is configured for attachment to a distal end of the urinary catheter both before and after use of the urinary catheter.

In some embodiments of the system, the urine collection bag is configured to be rolled up from the bottom end to the top end to transition the urine collection bag from the usage state to the storage state. In some embodiments of the system, the urinary catheter is pre-attached to the urine collection bag.

In some embodiments of the system, the storage bag is separably attached to bottom end of the urine collection bag. In some embodiments of the system, the storage bag is separably attached to the urine collection bag along a tear line. In some embodiments of the system, the storage bag is separably attached to either one of a front panel or a back panel of the urine collection bag.

In some embodiments of the system, the storage bag includes a closure mechanism configured to securely contain the urine collection bag within the storage bag. In some embodiments of the system, the closure mechanism includes a displaceable sealing member configured to define a fluid tight seal of storage bag opening when the displaceable sealing member is displaced across the storage bag opening.

In some embodiments of the system, the urine collection bag includes a handle disposed along a perimeter edge of the urine collection bag, and the handle includes a finger loop. In some embodiments of the system, the handle extends away from perimeter edge. In some embodiments of the system, the handle is disposed along a bottom perimeter edge of the urine collection bag.

In some embodiments of the system, the hub includes a one way valve disposed in line with the lumen of the hub, where the one way valve is configured to allow the urine to flow into the urine collection bag via the lumen of the hub and prevent the urine from flowing out of the urine collection bag via the lumen of the hub.

In some embodiments of the system, the hub includes a first hub portion coupled with the urine collection bag and a second hub portion coupled with the urinary catheter. The first and second hub portions define a continuity of the lumen of the hub when the first and second hub portions are coupled together. In some embodiments of the system, the second hub portion is detachably coupled with the first hub portion. In some embodiments of the system, the second hub portion is coupled with the first hub portion via a hinge so that rotation of the second hub portion with respect to the first hub portion transitions the first and second hub portions between a coupled state and a decoupled state. In some embodiments of the system, the first hub portion includes side port in fluid communication with the lumen of the hub. The first and second hub portions are slidably couple together so that the second hub portion is slidable between a depressed state with respect to the first hub portion and an extended state with respect to the first hub portion such that (i) when the second hub is in the extended state, the side port is in fluid communication with the lumen of the hub, thereby allowing urine to flow out of the urine collection bag via the side port, and (ii) when the second hub is in the depressed state, the side port is not in fluid communication with the lumen of the hub, thereby preventing urine from flowing out of the urine collection bag via the side port.

In some embodiments of the system, the urine collection bag is longitudinally extendable between the storage state and the usage state. In some embodiments of the system, the urine collection bag is longitudinally collapsed in the storage state.

In some embodiments of the system, the urine collection bag includes one or more straps configured to wrap around the urine collection bag in the storage state. In some embodiments of the system, the one or more straps extend away from a perimeter edge of the urine collection bag. In some embodiments of the system, each of the one or more straps includes one or more securing apertures extending through the respective strap, where the one or more securing apertures are configured to threadably receive the respective strap therethrough to secure the respective strap. In some embodiments of the system, the one or more straps includes an attachment device including a snap fastener, or a hook and loop fastener.

In some embodiments of the system, the pull ring of the catheter cap is configured to stretch so as to enable to the pull ring to extend around the urine collection bag in the storage state. In some embodiments of the system, the urine collection bag includes a rod extending along a bottom perimeter of the urine collection bag, and the rod enables the user to roll up the urine collection bag from the bottom end to the top end.

In some embodiments of the system, the outlet includes a frangible band extending across a corner perimeter of the urine collection bag. The frangible band includes pull tab configured to tear the frangible band when pulled by the user, where tearing the frangible band breaches a seal of the corner perimeter defining an outlet opening of the urine collection bag, and where the outlet opening allows for drainage of the urine from the urine collection bag. In some embodiments of the system, the frangible band is tethered to the urine collection bag.

In some embodiments of the system, the outlet includes (i) an outlet port defining an outlet port opening in fluid communication with an interior of the urine collection bag and (ii) an outlet cap sealably coupled with the outlet port, where the outlet cap is configured for separation from the outlet port to allow for drainage of the urine from the urine collection bag via the outlet port opening. In some embodiments of the system, the outlet cap is hingedly coupled with the outlet port. In some embodiments of the system, the cap is threadably coupled with the outlet port.

In some embodiments of the system, outlet includes an outlet port defining an outlet port opening in fluid communication with an interior of the urine collection bag, where the outlet port includes a frangible film extending across the outlet port opening, and where the frangible film defines a film seal of the outlet port opening. In some embodiments of the system, the frangible film is a paper material configured to dissolve upon contact with urine such that, during use, contact of the urine with the paper material breaches the film seal allowing for drainage of the urine from the urine collection bag via the outlet port opening. In some embodiments of the system, the outlet port includes a piercing member tethered thereto, where the piercing member is configured to pierce the frangible film, thereby breaching the film seal to allow for drainage of the urine from the urine collection bag via the outlet port opening. In some embodiments of the system, the piercing member includes a piercing member body defining a lumen of the piercing member, the piercing member configured to couple with the outlet port so that the lumen of the piercing member is in fluid communication with the outlet opening.

In some embodiments of the system, the outlet includes a first pull tab coupled with the front panel and a corresponding second pull tab coupled with the back panel, where each of the first and second pull tabs extend away from a corner perimeter of the urine collection bag, where in use, pulling the first and second pull tabs apart from each other breaches a seal of the corner perimeter defining an outlet opening of the urine collection bag to allow for drainage of the urine from the urine collection bag via the outlet opening. In some embodiments of the system, each of the first and second pull tabs includes a finger aperture.

Also disclosed herein is a urine collection bag that, according to some embodiments, includes a top end and a bottom end. The bag further includes a hub at the top end configured to couple with a urinary catheter. The urine collection bag further includes (i) an inlet opening coupled with the hub, where the inlet opening is in fluid communication with a lumen the hub and (ii) an outlet configured to transition from a closed state to an opened state to allow urine to be drained from the urine collection bag. The urine collection bag further includes a storage bag separably attached to the urine collection bag, where the storage bag is configured to receive therein the urinary catheter and the urine collection bag in a storage state via an opening of the storage bag.

In some embodiments of the bag, the urine collection bag is configured to be rolled up from a distal end to a proximal end to transition the urine collection bag from the usage state to the storage state.

In some embodiments of the bag, the storage bag is separably attached to the bottom end of the urine collection bag. In some embodiments of the bag, the storage bag is separably attached to the urine collection bag along a tear line. In some embodiments of the bag, the storage bag is separably attached to either one of a front panel or a back panel of the urine collection bag.

In some embodiments of the bag, the storage bag includes a closure mechanism configured to securely contain the urine collection bag within the storage bag. In some embodiments of the bag, the closure mechanism includes a displaceable sealing member configured to define a fluid tight seal of storage bag opening when the displaceable sealing member is displaced across the storage bag opening.

In some embodiments of the bag, the urine collection bag includes a handle disposed along a perimeter edge of the urine collection bag, where the handle includes a finger loop. In some embodiments of the bag, the handle extends away from perimeter edge. In some embodiments of the bag, the handle is disposed along a bottom perimeter edge of the urine collection bag.

In some embodiments of the bag, the hub includes a one way valve disposed in line with the lumen of the hub, the one way valve configured to allow urine to flow into the urine collection bag via the lumen of the hub and prevent urine from flowing out of the urine collection bag via the lumen of the hub.

In some embodiments of the bag, the hub includes a first hub portion coupled with the urine collection bag and a second hub portion configured to couple with the urinary catheter. The first and second hub portions define a continuity of the lumen of the hub when the first and second hub portions are coupled together. In some embodiments of the bag, the second hub portion is detachably coupled with the first hub portion. In some embodiments of the bag, the second hub portion is coupled with the first hub portion via a hinge so that rotation of the second hub portion with respect to the first hub portion transitions the first and second hub portions between a coupled state and a decoupled state.

In some embodiments of the bag, the first hub portion includes side port in fluid communication with the lumen of the hub. The first and second hub portions are slidably couple together so that the second hub portion is slidable between a depressed state with respect to the first hub portion and an extended state with respect to the first hub portion. In such embodiments, when the second hub is in the extended state, the side port is in fluid communication with the lumen of the hub, thereby allowing the urine to flow out of the urine collection bag via the side port, and when the second hub is in the depressed state, the side port is not in fluid communication with the lumen of the hub, thereby preventing the urine from flowing out of the urine collection bag via the side port.

In some embodiments of the bag, the urine collection bag is longitudinally extendable between the storage state and the usage state. In some embodiments of the bag, the urine collection bag is longitudinally collapsed in the storage state.

In some embodiments of the bag the urine collection bag includes one or more straps configured to wrap around the urine collection bag in the storage state. In some embodiments of the bag, the one or more straps extend away from a perimeter edge of the urine collection bag. In some embodiments of the bag, each of the one or more straps includes one or more securing apertures extending through the respective strap, and the one or more securing apertures are configured to threadably receive the respective strap therethrough to secure the respective strap. In some embodiments of the bag, the one or more straps include an attachment device including a snap fastener, or a hook and loop fastener.

In some embodiments of the bag, the urine collection bag includes a rod extending along a bottom perimeter of the urine collection bag, and the rod enables the user to roll up the urine collection bag from the bottom end to the top end.

In some embodiments of the bag, the outlet includes a frangible band extending across a corner perimeter of the urine collection bag. The frangible band includes pull tab configured to tear the frangible band when pulled by the user, where tearing the frangible band breaches a seal of the corner perimeter defining an outlet opening of the urine collection bag, and where the outlet opening allows for drainage of the urine from the urine collection bag. In some embodiments of the bag, wherein the frangible band is tethered to the urine collection bag.

In some embodiments of the bag, the outlet includes (i) an outlet port defining an outlet port opening in fluid communication with an interior of the urine collection bag; and (ii) an outlet cap sealably coupled with the outlet port, where the outlet cap is configured for separation from the outlet port to allow for drainage of the urine from the urine collection bag via the outlet port opening. In some embodiments of the bag, the outlet cap is hingedly coupled with the outlet port. In some embodiments of the bag, the cap is threadably coupled with the outlet port.

In some embodiments of the bag, the outlet includes an outlet port defining an outlet port opening in fluid communication with an interior of the urine collection bag, where the outlet port includes a frangible film extending across the outlet port opening, and where the frangible film defines a film seal of the outlet port opening. In some embodiments of the bag, the frangible film is a paper material configured to dissolve upon contact with urine such that, during use, contact of the urine with the paper material breaches the film seal allowing for drainage of the urine from the urine collection bag via the outlet port opening. In some embodiments of the bag, the outlet port includes a piercing member tethered thereto, where the piercing member is configured to pierce the frangible film, thereby breaching the film seal to allow for drainage of the urine from the urine collection bag via the outlet port opening. In some embodiments of the bag, the piercing member includes a piercing member body defining a lumen of the piercing member, where the piercing member is configured to couple with the outlet port so that the lumen of the piercing member is in fluid communication with the outlet opening.

In some embodiments of the bag, the outlet includes a first pull tab coupled with the front panel and a corresponding second pull tab coupled with the back panel, where each of the first and second pull tabs extend away from a corner perimeter of the urine collection bag, and where, in use, pulling the first and second pull tabs apart from each other breaches a seal of the corner perimeter defining an outlet opening of the urine collection bag to allow for drainage of the urine from the urine collection bag via the outlet opening. In some embodiments of the bag, each of the first and second pull tabs includes finger aperture.

These and other features of the concepts provided herein will become more apparent to those of skill in the art in view of the accompanying drawings and following description, which describe particular embodiments of such concepts in greater detail.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A illustrates a plan view of a urinary catheter system in a pre-use storage state within a package, in accordance with some embodiments;

FIG. 1B illustrates a plan view of the system of FIG. 1A removed from the package, in accordance with some embodiments;

FIG. 1C illustrates a plan view of the system of FIG. 1B in a ready-to-use state, in accordance with some embodiments;

FIG. 2 illustrates the system of FIGS. 1A-1C in use, in accordance with some embodiments;

FIG. 3 illustrates a plan view of an embodiment of a fluid collection bag of the system of FIGS. 1A-1C, in accordance with some embodiments;

FIGS. 4A-4F illustrate perspective views of different embodiments of an inlet opening of the bag of the FIGS. 1A-1C, in accordance with some embodiments;

FIGS. 5A-5K illustrate perspective views of various embodiments of an outlet of the bag of the FIGS. 1A-1C, in accordance with some embodiments;

FIGS. 6A-6T illustrate perspective views of various embodiments of the bag of the FIGS. 1A-1C transitioning from the usage state to the storage state, in accordance with some embodiments; and

FIGS. 7A-7F illustrates a plan view another embodiment of the bag of the FIGS. 1A-1C transitioning from the usage state to the storage state, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

The phrases “connected to,” “coupled with,” and “in communication with” refer to any form of interaction between two or more entities, including but not limited to mechanical, and fluid interaction. Two components may be coupled with each other even though they are not in direct contact with each other. For example, two components may be coupled to each other through an intermediate component.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

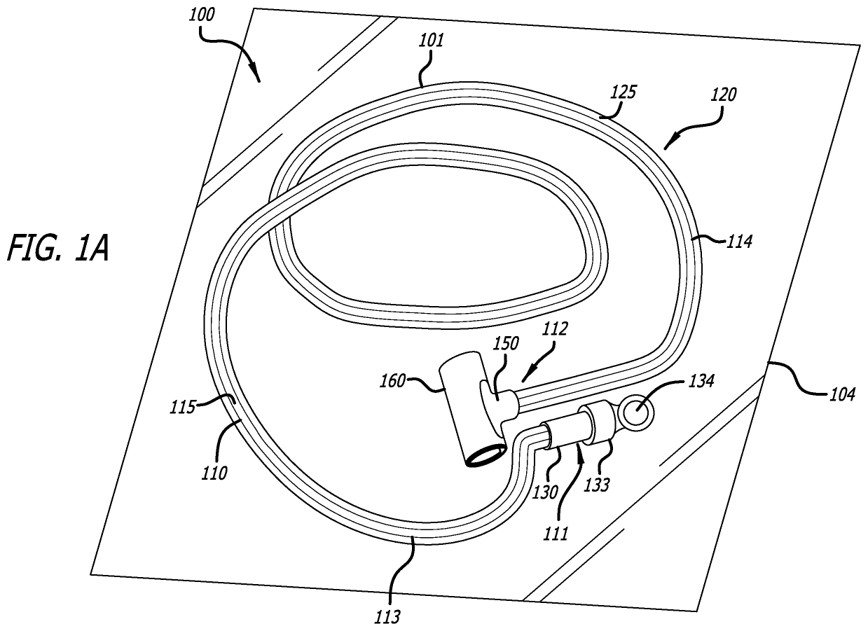

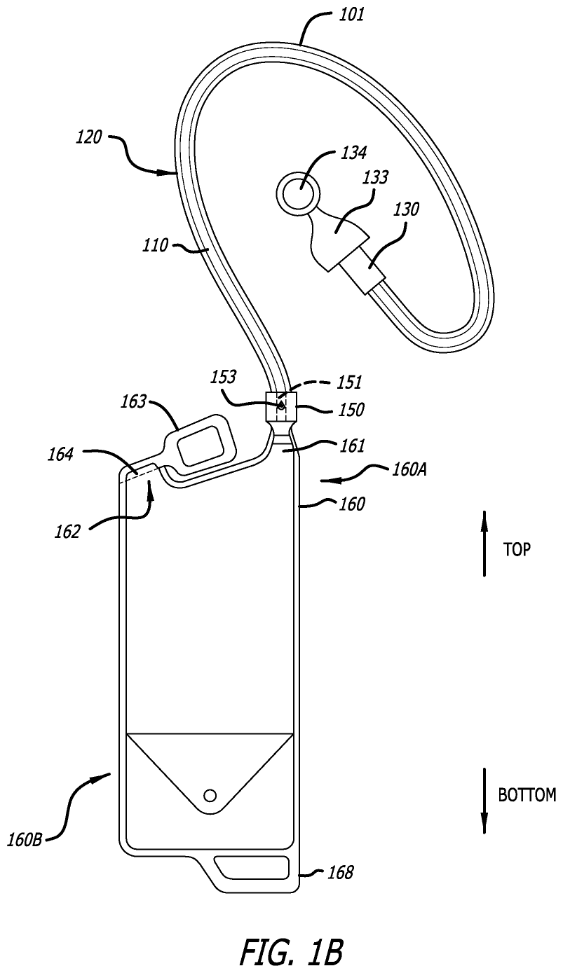

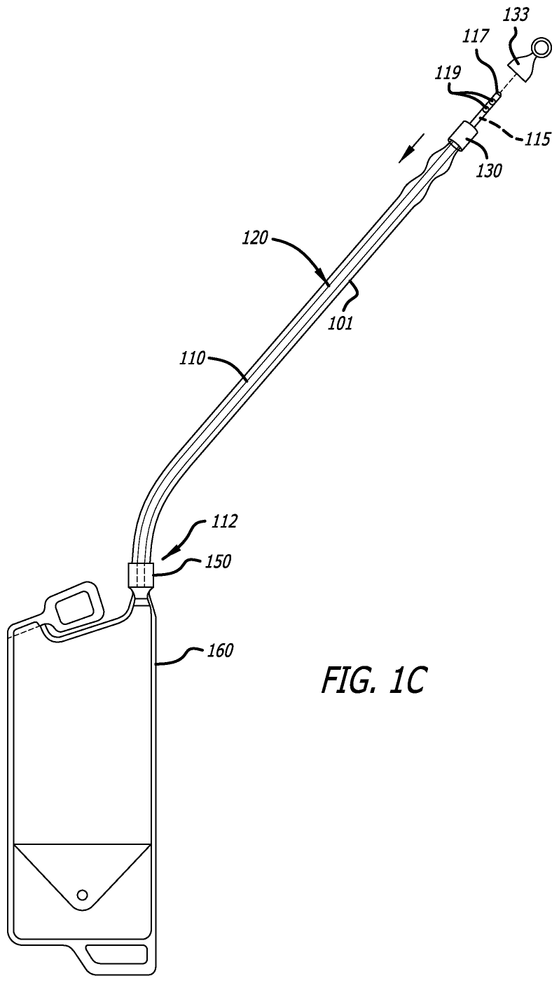

FIG. 1A illustrates a perspective front view of the system 100 in a pre-use packaging state. The system 100 may be disposed in the pre-use packaging state before use. The system 100 may be disposed within an outer package 104, such as a bag or pouch, for example. In some embodiments, the outer package 104 may be resealable. In other words, the outer package 104 may be configured to (i) sealably contain the system 100 before use and (ii) sealably contain the system 100 after use.

The system 100 generally includes a urinary catheter (catheter) 101 and a collection bag (bag) 160. During various stages of use, the bag 160 and the catheter 101 are coupled together. The catheter 101 generally includes a catheter tube 110 having a proximal end 112 and a distal end 111. The catheter tube 110 may be formed of any suitable thermoplastic material via an extrusion process. A catheter lumen 115 extends along the catheter tube 110, where the catheter lumen 115 is sized to facilitate urine flow therethrough from the distal end 111 to the proximal end 112 during use. The catheter tube 110 includes an inlet section 113 extending proximally away from the distal end 111, and the inlet section 113 is configured for insertion into and advancement along a urethra of the user to the user's bladder. As such, the inlet section 113 defines a length sufficient to extend from the end of a penis to the bladder of the user. Similarly, the inlet section 113 defines an outside diameter compatible with placement within the urethra. The catheter tube 110 also includes an outlet section 114 extending distally away from the proximal end 112. The outlet section 114 is configured for use outside of the user's body.

The catheter 101 includes an inlet collar 130 coupled with the inlet section 113 to provide an insertion aid to the user. The inlet collar 130 is configured for gripping by the user during use. In some embodiments, the inlet collar 130 includes a cap (or plug) 133 that is configured to seal or close off the distal end 111 of the catheter 101 in the pre-use packaging state as further described below. In some embodiments, the cap 133 may include a pull ring 134 to facilitate removal of the cap 133 from the inlet collar 130 to break the seal. In some embodiments, the cap 133 may be configured for recoupling to the inlet collar 130 to reseal the distal end 111. The inlet collar 130 may also include an attachment member 135 as further described below.

The catheter 101 also includes an outlet hub (hub) 150 defining a handle for the catheter 101 during use. The hub 150 is attached to the catheter tube 101 at the proximal end 112. The hub 150 includes may include gripping features (not shown) such as protrusions, depressions, ribs, or toughs, for example, disposed on an outside surface of the hub 150 to enable grasping of the hub 150 during use. A lumen of the hub 150 provides for fluid communication between the catheter lumen 115 and an interior of the bag 160.

The system 100 further includes a sheath 120 extending along the catheter tube 110 from the inlet collar 130 to the hub 150. The sheath 120 defines a sheath lumen 125 and the catheter tube 110 is disposed within the sheath lumen 125. The sheath 120 (or more specifically a distal end of the sheath 120) is sealably attached to the inlet collar 130 and the sheath 120 is also sealably attached to the hub 150.

The bag 160 is folded or otherwise formed into a compact shape for placement within the outer package 104. The compact shape may also facilitate placement of the system 100 within the pocket or purse of the user.

FIG. 1B illustrates the system 100 in an unpackaged state, i.e., where the system 100 is removed from the outer package 104. The bag 160 in unfolded from the pre-use packaging state. The bag 160 is configured to receive therein urine output from the user. As shown above, the bag 160 may be collapsible, i.e., the bag 160 may be configured to transition between a storage state (e.g., the pre-use packaging state) and a usage state, as will be described in more detail herein.

The bag 160 includes a first opening 161. The outlet port 150 is be coupled to the first opening 166 and an outlet hub lumen 151 provides for urine flow from the catheter lumen 115 to the first opening 161. In some embodiments, the first opening 161 may be located at the top end 160A of the bag 160. In some embodiments, the outlet hub 150 may include a one way valve 152 disposed in line with the outlet hub lumen 151, where the one way valve 152 is configure to (i) allow urine to flow into the bag 160 and (ii) prevent urine from flowing out of the bag 160 via the outlet hub lumen 151.

The bag 160 further includes an outlet 130 configured to allow for draining of the urine from the bag 160. The outlet 130 is configured to transition between (i) a closed state preventing urine flow through the outlet 162 and (ii) an opened state allowing urine flow through the outlet 130. In some embodiments, the outlet 162 may be located at the top end 160A of the bag 160, such as adjacent to the first opening 161, for example. In some embodiments, the outlet 162 may be pre-disposed to the closed state. In some embodiments, the user may transition the outlet 162 from the closed state to the opened state as will be described in more detail herein. In some embodiments, the outlet 162 may include a pull tab 163 coupled to a frangible band 164, where the frangible band 164 is configured to transition outlet 162 from the closed state to the opened state. In some embodiments, the bag 160 may include a handle 168 located at a bottom end 160B of the bag 160. In some embodiments, the handle 168 may be located at the top end 160A or on a side of the bag 160. The handle 118 may enable the user to empty the urine from the bag 160. In some embodiments, the handle 168 may extend away from perimeter edge of the bag 160, such as the perimeter edge at the bottom 160B of the bag 160, for example.

FIG. 1C illustrates the system 100 in a ready-to-use state. In the ready-to-use state, the catheter tube 110 is uncoiled, the bag 160 is unfolded, and the cap 133 is removed from the inlet collar 130. The inlet collar 130 is configured to slide along the catheter tube 110 away from the distal end 111. In the ready-to-use state, the inlet collar 130 is proximally displaced to expose a catheter tip 117. The catheter tip 117 includes a number (e.g., 1, 2, or more) of eyelets 119 of the catheter tube 110. The eyelets 119 define a distal opening of the catheter lumen 115 to allow urine to enter the catheter lumen 115 during use.

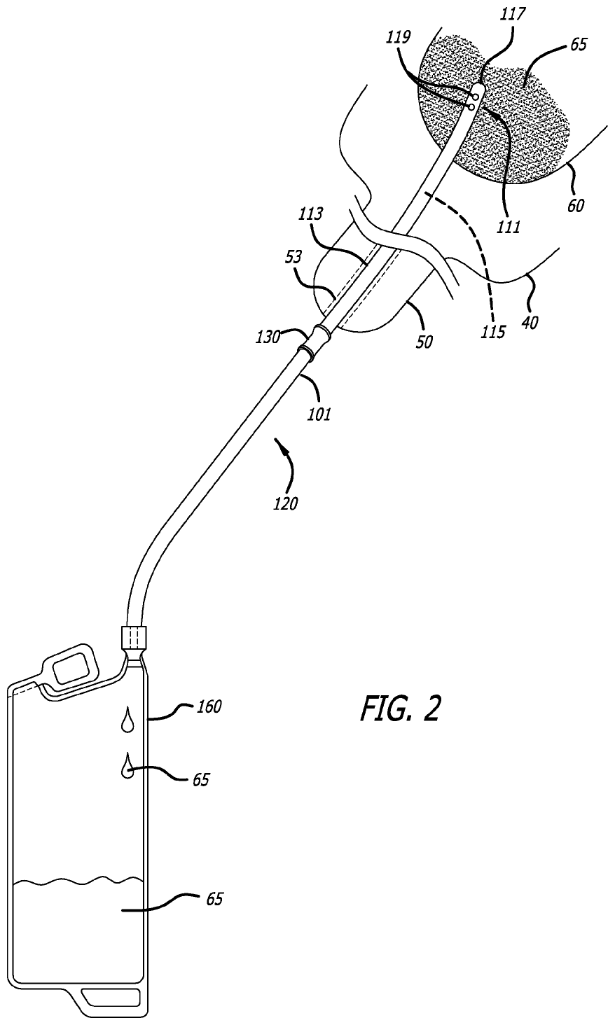

FIG. 2 illustrates the system 100 during use. The inlet collar 130 is proximally displaced to expose the inlet section 113 and the inlet section 113 is advanced through the urethra 53 of the penis 50 so that the distal end 111 is disposed within the bladder 60 of the user 40. Urine 65 passes through the eyelets 119 into the catheter lumen 115. The urine 65 then flows from the catheter lumen 115 into the bag 160. It is noted that, although the system 100 is shown and described for use with a male user (or patient), the system may in some embodiments be configured for use with a female user (or patient).

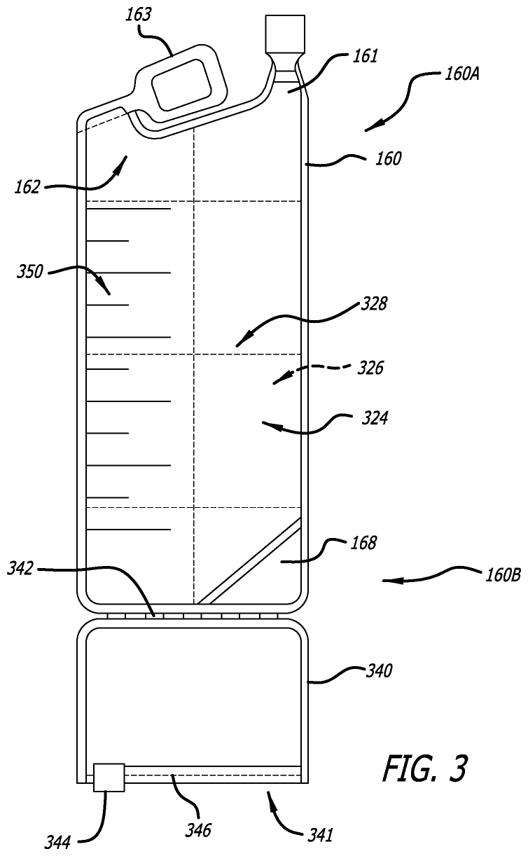

FIG. 3 is a plan view of the bag 160 illustrating further details of the bag 160. In some embodiments, the bag 160 may include a storage bag 340 configured to receive and store the bag 160 therein. In some embodiments, the storage bag 340 may be detachably coupled to the bag 160. In some embodiments, the storage bag 340 may include a storage bag opening 341. In some embodiments, the storage bag 340 may be detachably coupled to a front panel 324 or a back panel 326 of the bag 160. In some embodiments, the storage bag 340 may include a securement mechanism configured to close the storage bag opening 341. In some embodiments, the storage bag 340 may be coupled to the bag 160 along one or more score lines 342. In some embodiments, the storage bag 340 includes a sealing member 344 configured to generate a liquid tight press seal 346 to seal the storage bag opening 341 when the sealing member 344 is displaced across the storage bag opening 341. In some embodiments, the storage bag 340 and receive therein the bag 160 in the storage state and the catheter 101.

In some embodiments, the bag 160 may include graduation lines 150 configured to indicate the volume of urine captured within the bag 160. In some embodiments, the graduation lines 350 may be printed on the front panel 324 or the back panel 326 of the bag 160. In some embodiments, the bag 160 may be configured to transition between the storage state and the usage state along a number of fold lines 328 wherein the fold lines 328 predispose the bag 160 to collapse, fold or roll into the storage state. In some embodiments, the fold lines 328 may be organized into one or more parallel transverse lines and/or one or more parallel longitudinal lines as illustrated in FIG. 3. In some embodiments, the fold lines 328 may include the parallel transverse lines bisecting the parallel longitudinal lines. In some embodiments, the fold lines 328 may be pressed or engraved into the bag 160. In some embodiments, the fold lines 328 may be pressed into the front panel 324, the back panel 326 or a combination thereof. In some embodiments, having the fold lines 328 pressed into the front panel 324 may bias the bag 160 to fold towards the front panel 324, whereas having the fold lines 328 pressed into the back panel 326 may bias the bag 160 to fold towards the back panel 326. In some embodiments, a first subset of the fold lines 328 may be pressed into the front panel 324 and a second non-intersecting subset of the fold lines 328 may be pressed into the back panel 326, allowing portions of the bag 160 to be biased towards the front panel 324 with portions of the bag 160 biased towards the back panel 326.

An exemplary method of use of the system 100 may include all or any subset of the following steps, actions, or processes. The user must choose a place (e.g., a bathroom stall) to void a volume of urine. The user may then sanitize their hands and place the system 100 on a clean surface. Wearing gloves, the user may open the packaging 104 of the system 100 and remove the bag 106 and catheter 101 from the package 104. The user may sanitize the head of the penis. The user may unfold the bag 160. The user may remove the cap 133 and advance the inlet section 113 along the urethra 53 until the tip 117 of the catheter tube 110 resides within the bladder 60. The user may then excrete urine into the bag 160 by way of the catheter tube 110. Once thew urine is excreted into the bag 160, the user may remove the inlet section 113 from the urethra 53 while maintaining control of the bag 160 so to prevent urine from flowing back into the catheter tube 110. The user may record the volume of urine collected. To dispose of the urine, the user may transition the outlet 162 from the closed state to the opened state and drain the collected urine into the toilet. The user may separate the storage bag 340 from the bag 160. The user may roll, fold or otherwise transition the bag 160 from the usage state to the storage state. The user may place the bag 160 and the catheter 101 into the storage bag 340. The user may displace the securement member 344 across the opening 341 of the storage bag 340 to sealably close off the storage bag 340.

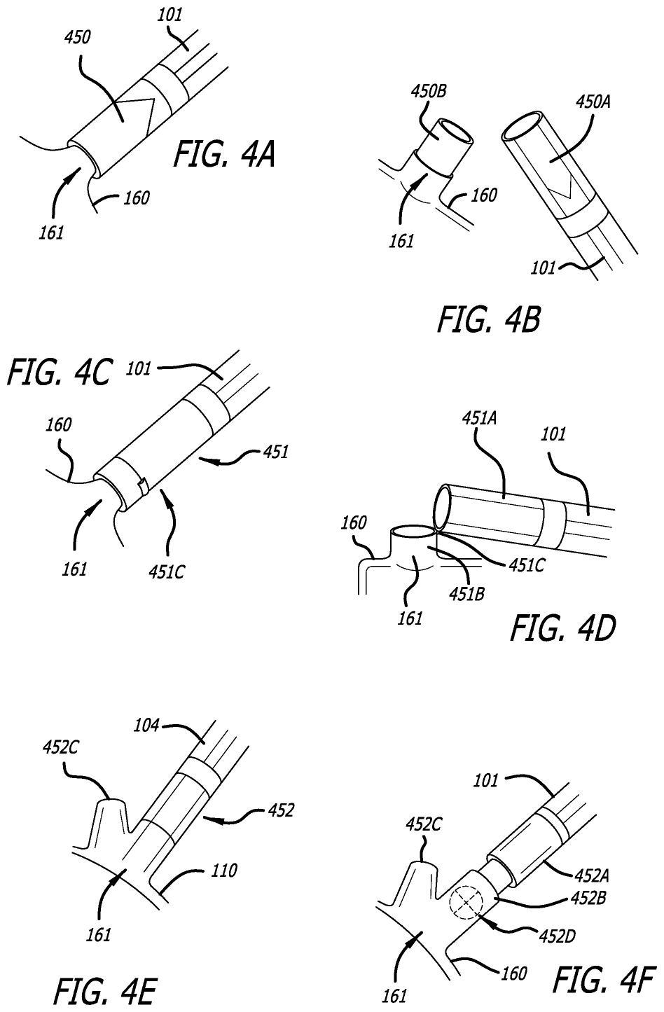

FIGS. 4A-4F illustrate perspective views of different embodiments of the first opening 161 and the outlet hub 150 where each of the illustrated embodiments may in some respects resemble the components and functionality of the opening 161 and the outlet hub 150.

FIGS. 4A-4B illustrate an outlet hub 450 including a first hub portion 450A coupled with the catheter 101 and a second hub portion 450B coupled with the first opening 161 of the bag 160. The first hub portion 450A is detachably coupled with the second hub portion 450B, such that (i) when the first and second hub portions 450A, 450B are coupled together (FIG. 4A), fluid communication is established between the catheter 101 and the bag 160 (i.e., the first and second hub portions 450A, 450B define a continuity of the lumen of the hub 450 when the first and second hub portions 450A, 450B are coupled together), and when the first and second hub portions 450A, 450B are decoupled from each other (FIG. 4B), the urine may be drained from the bag via the first opening 161 and the second hub portion 450B. In some embodiments, the second hub portion 450B may define rigid nozzle as illustrated in FIG. 4B. In some embodiments, the first and second hub portions 450A, 450B may be coupled to each other via a snap fit.

FIGS. 4C-4D illustrate an outlet hub 451 including a first hub portion 451A coupled with the catheter 101 and a second hub portion 451B coupled with the first opening 161 of the bag 160. The first hub portion 451A is hingedly coupled with the second hub portion 451B via a hinge 451C, such that (i) when the first and second hub portions 451A, 451B are hingedly closed (FIG. 4C), fluid communication is established between the catheter 101 and the bag 160, and when the first and second hub portions 451A, 451B are hingedly opened (FIG. 4B), the urine may be drained from the bag 160 via the first opening 161 and second hub portion 451B. In some embodiments, the second hub portion 451B may define rigid nozzle as illustrated in FIG. 4D. In some embodiments, the first and second hub portions 451A, 451B may be secured in the hingedly closed state via a snap fit.

FIGS. 4E-4F illustrate an outlet hub 452 including a first hub portion 452A coupled with the catheter 101 and a second hub portion 452B coupled with the first opening 161 of the bag 160. The first hub portion 452A is slidably coupled with the second hub portion 452B, such that (i) when the first and second hub portions 452A, 452B are slidably depressed (FIG. 4E), fluid communication is established between the catheter 101 and the bag 160, and when the first and second hub portions 452A, 452B are slidably extended (FIG. 4B), a valve 452D is opened so that urine may be drained from the bag 160 via a side port 452C of the second hub portion 452B. In some embodiments, the side port 452C may define rigid nozzle as illustrated in FIG. 4F. In some embodiments, the first and second hub portions 452A, 452B may be secured in the slidably depressed state via snap fit.

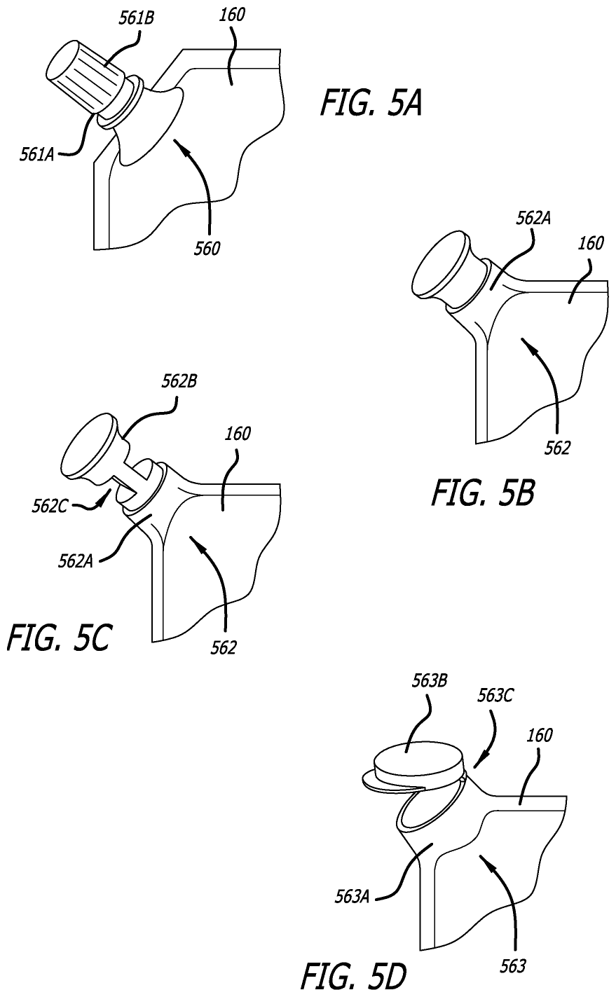

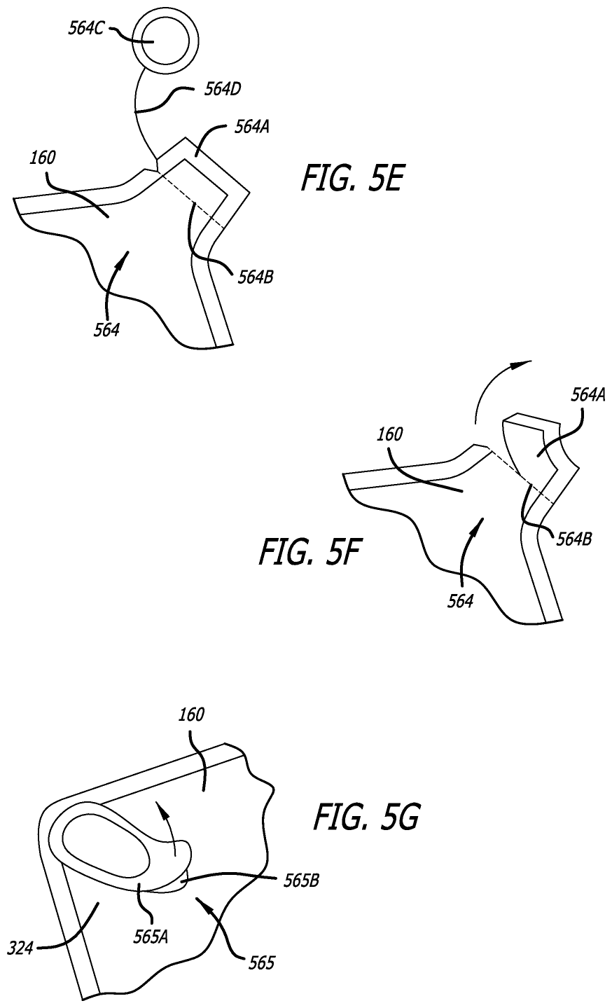

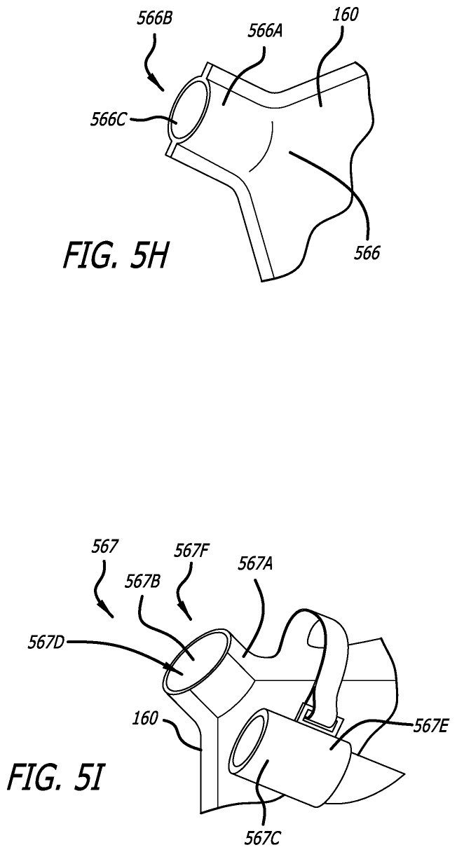

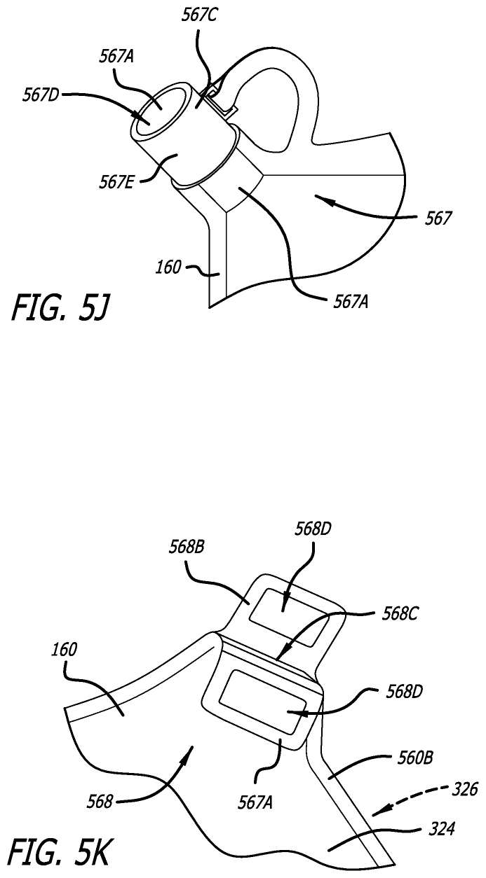

FIGS. 5A-5K illustrate perspective views of various embodiments of the outlet 130 of the bag 160 where each of the illustrated embodiments may in some respects resemble the components and functionality of the outlet 162.

FIG. 5A illustrates an outlet 560 that may transition from the closed state to the opened state to drain urine from the bag 160. The outlet 560 includes a nozzle 561A having a cap 561B removably coupled with the nozzle 561A, as illustrated in FIG. 5A. In some embodiments, the cap 561B may be threadably coupled with the nozzle 561A. During use, the cap 561B is removed from the nozzle 561A to allow drainage of the urine from the bag 160.

FIGS. 5B-5C illustrate an outlet 562 including a first outlet portion 562A with the bag 160 and a second outlet portion 562B coupled the first outlet portion 562A. The second portion 562B is slidably coupled with the first outlet portion 562A, such that (i) when the first and second outlet portions 562A, 562B are slidably depressed (FIG. 5B), the outlet 562 is transitioned to the closed state, and when the first and second outlet portions 562A, 562B are slidably extended (FIG. 5C), the urine may be drained from the bag 160 via a side port 562C. In some embodiments, the first and second outlet portions 562A, 562B may be secured in the slidably depressed state via a snap fit.

FIG. 5D illustrates an outlet 563 including an outlet nozzle 563A coupled with the bag 160 and an outlet cap 563B hingedly coupled with the outlet nozzle 563A via a hinge 563C, such that (i) when outlet cap 563B secured to the outlet nozzle 563A, urine drainage through the outlet nozzle 563A is prevented, and when outlet cap 563B is hingedly separated from the outlet nozzle 563A, the urine may be drained from the bag 160 via the outlet nozzle 563A. In some embodiments, the outlet nozzle 563A may define rigid nozzle as illustrated. In some embodiments, the outlet cap 563B may be secured to outlet nozzle 563A via a snap fit.

FIGS. 5E-5F illustrate an outlet 564 that includes the frangible band 564A covering the opening of the outlet 564 (FIG. 5E). The frangible band 564A is configured to be torn along a frangible line 564B, where the frangible line 564B may be disposed diagonally across a corner of the bag 160. In some embodiments, the frangible band 564A may be completely removed from the bag 160 to transition the outlet 564 from the closed state to the opened state (FIG. 5F). In some embodiments, the frangible band 564A may include a pull tab 564C. In some embodiments, the pull tab 132 may be coupled to a string 564D running along the frangible line 564B. The pull tab 564C may be pulled, allowing the string 564D to separate the frangible band 564A from the bag 160 along the frangible line 564B to transition the outlet 564 from the closed state to the opened state. In some embodiments, the frangible band 564A may be tethered to the bag 160 so that when the frangible band 564A is torn from the bag 160 along the frangible line 564B, the frangible band 564A remains attached to the bag 160.

FIG. 5G illustrates an outlet 565 the includes frangible band 565A covering an opening 565B extending through the front panel 324 of the fluid collection bag 160. In some embodiments, the frangible band 565A may be pulled away from the front panel 324 to uncover the opening 565B to transition the outlet 565 from the closed state to the opened state.

FIG. 5H illustrates an outlet 566 that includes a nozzle 566A coupled with and extending from the bag 160. The nozzle 566A defines a nozzle opening 566B covered by frangible paper material 566C. The frangible paper material 566C may be dissolvable when the frangible paper material 566C comes into contact with a liquid (e.g., urine, water, or the like). The liquid may dissolve the frangible paper material 566C so as to uncover the nozzle opening 566B, transitioning the outlet 566 from the closed state to the opened state.

FIGS. 51-5J illustrate an outlet 567 including a piercing member 567C that may be tethered to a nozzle 567A coupled with the bag 160. A user may use the piercing member 567C to pierce a frangible film 567B covering the nozzle opening 567F, thereby uncovering the nozzle opening 567F to transition the outlet 567 from the closed state to the opened state. In some embodiments, the piercing member 567C may include a piercing member body 567E defining a lumen 567D extending therethrough. The user may pierce the nozzle opening 567F using the piercing member 567C, leaving the piercing member 567C within the nozzle 567A to allow urine to drain through the lumen 567D out of the bag 160, as illustrated in FIG. 5J.

FIG. 5K illustrates an outlet 568 that includes two opposing pull tabs 568A, 568B. The pull tabs 568A, 568B are coupled with the front panel 324 and the back panel 326, respectively. The seam 560B of the bag 160 extends across the outlet 568 to define a closed state of the outlet 568. The opposing pull tabs 568A, 568B are pulled apart to separate the seam 560B defining an opening 568C through which the urine may be drained from the bag 160. The pull tabs 568A, 569B may each include a finger loop 568D.

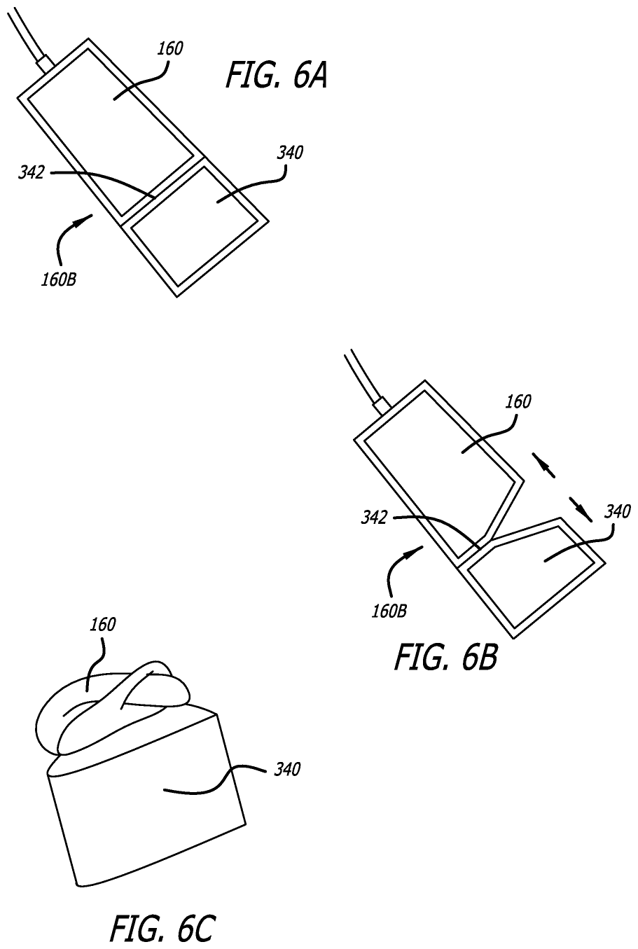

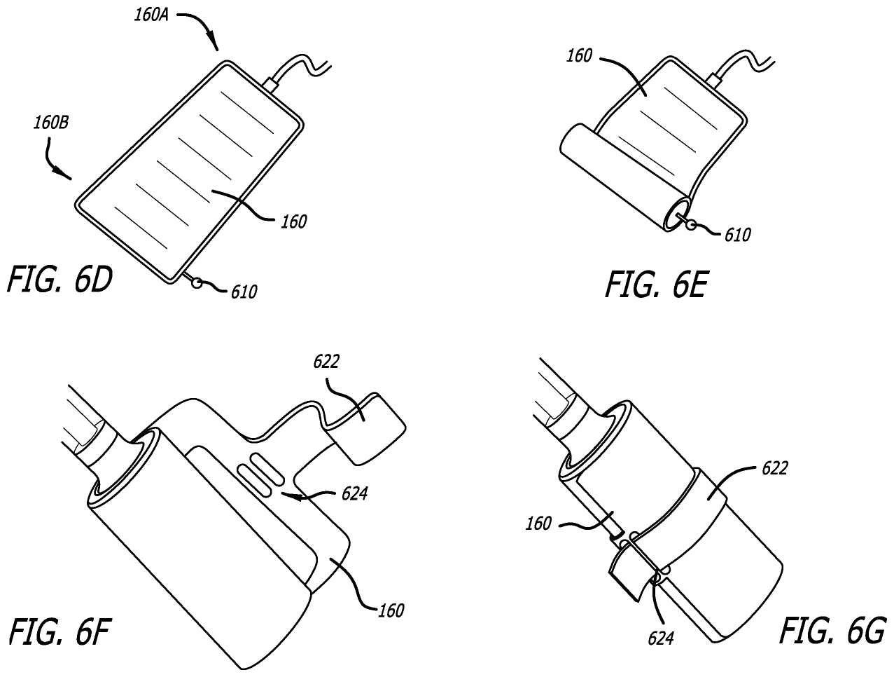

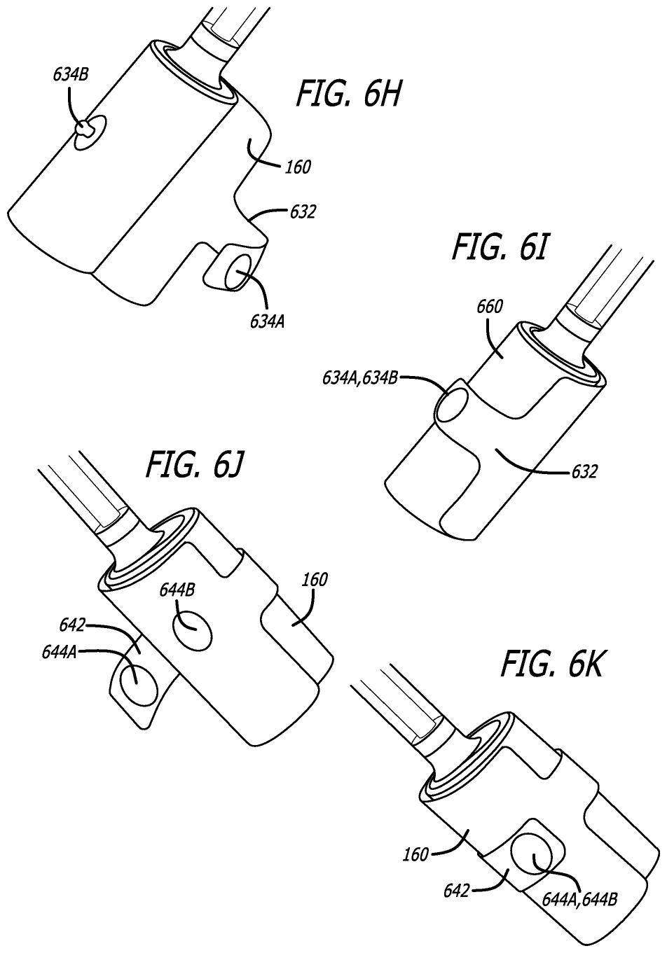

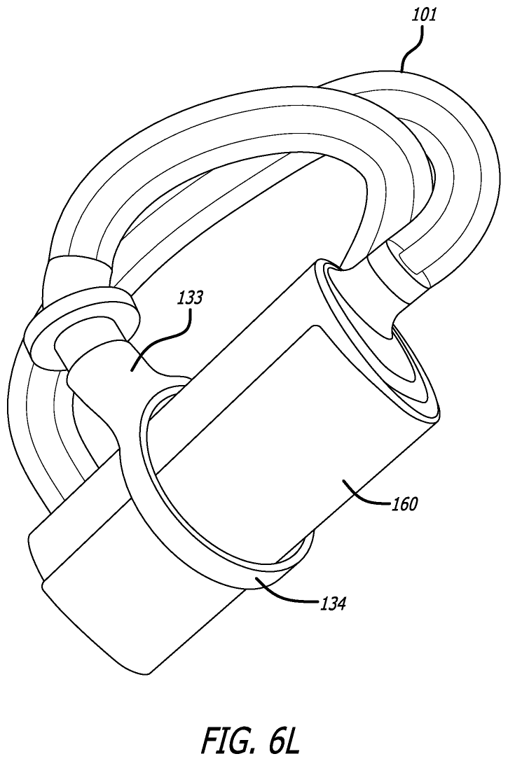

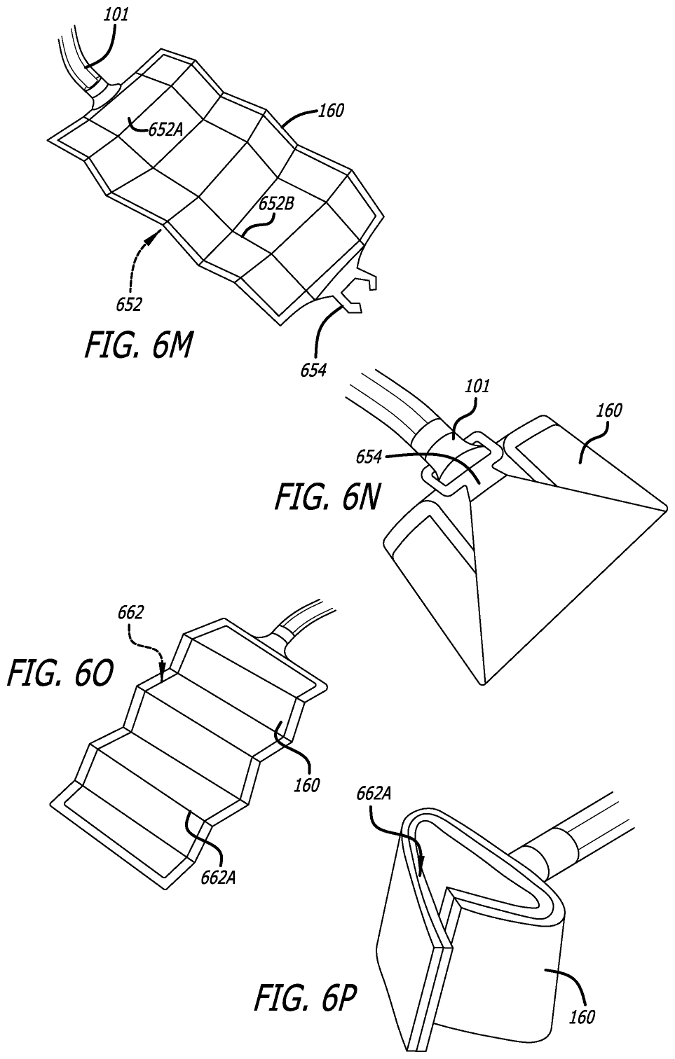

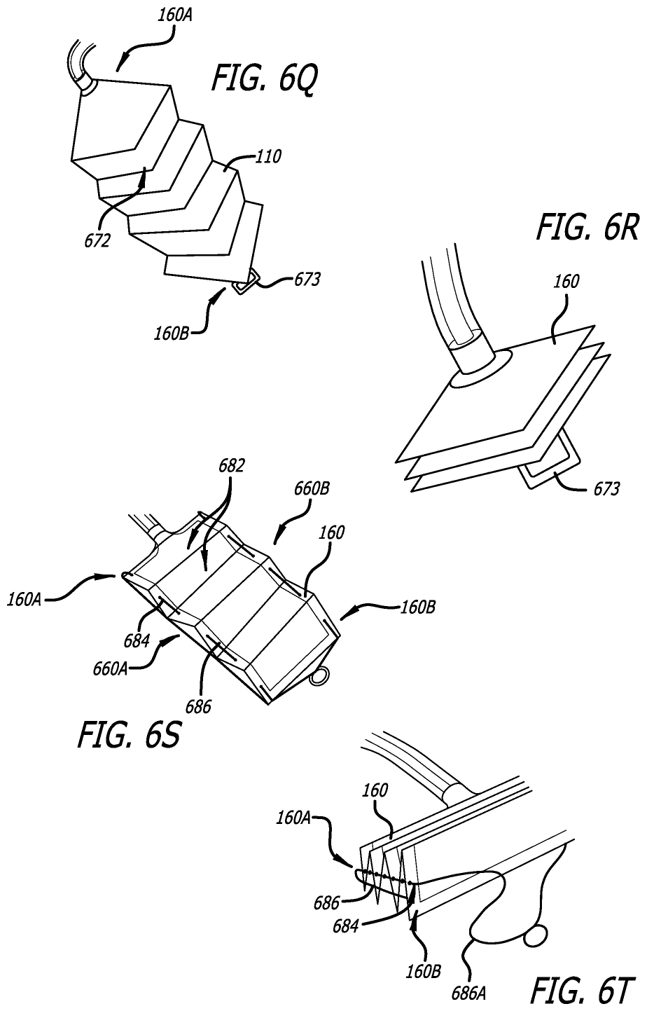

FIGS. 6A-6T illustrate perspective views of different methods of transitioning the bag 160 from the usage state to the storage state. As illustrated in FIGS. 6A-6C, the bag 160 may include the storage bag 340 detachably coupled to the bottom end 160B of bag 160 in the usage state (FIG. 6A). The storage bag 340 may be detached from the bag 160 along the score line 342 (FIG. 6B). The bag 160 may be collapsed (e.g., folded, rolled, or compressed) from the usage state to the storage state so that the bag 160 may be placed into the storage bag 340 (FIG. 6C.

FIGS. 6D-6E illustrate how the bag 160 may be rolled from the bottom end 160B to the top end 160A to transition the bag 160 from the usage state to the storage state. In some embodiments, the bag 160 may be rolled from the bottom end 160B to the top end 160A to assist in emptying the bag 160 from the urine collected therein. The bag 160 includes rod 610, extending laterally away from the bag 160 (FIG. 6D). In some embodiments, the rod 610 may be coupled with the bag 160 along a perimeter disposed along the bottom end 160B of the bag 160. The user may use the rod 610 to assist in rolling the bag 160 into the storage state (FIG. 6E). In some embodiments, the user may rotate the rod 610 clockwise to roll up the bag 160.

FIGS. 6F-6G illustrate a first embodiment of a securement mechanism to maintain the bag 160 in the storage state (e.g., rolled up). The bag 160 includes a strap 622 extending away from a perimeter edge of the bag 160. In some embodiments, the strap 622 may extend transversely away from the bag 160 adjacent the bottom end 160B or the top end 160A. The bag 160 may be rolled in the direction of the strap 622. The strap 622 includes securing apertures 624, configured to enable the strap 622 to be wrapped around the bag 160 in the storage state and looped through the securing apertures 624 to secure the bag 160 in the storage state (FIG. 6G).

FIGS. 6H-6I illustrate a second embodiment of a securement mechanism to maintain the bag 160 in the storage state (e.g., rolled up). The bag 160 includes a strap 632 extending away from a perimeter edge of the bag 160. In some embodiments, the strap 632 may extend transversely away from the bag 160 adjacent the bottom end 160B or the top end 160A. The bag 160 may be rolled in the direction of the strap 632. The securement mechanism includes a snap fastener including a first portion 634A of the snap fastener coupled with the strap 632 and a corresponding second portion 634B couple with the bag 160. When the bag 160 is in the storage state, the first and second portions 634A, 634B may be coupled together to secure the bag 160 in the storage state (FIG. 6I). In some embodiments, the securement mechanism may include a button, a hook, or a buckle in leu of the snap fastener. In some embodiments, the bag 160 may include more than one strap 632.

FIGS. 6J-6K illustrate a third embodiment of a securement mechanism to maintain the bag 160 in the storage state (e.g., rolled up). The bag 160 includes a strap 642 extending away from a perimeter edge of the bag 160. In some embodiments, the strap 642 may extend transversely away from the bag 160 adjacent the bottom end 160B or the top end 160A. The bag 160 may be rolled in the direction of the strap 642. The securement mechanism includes a hook and loop fastener including a first hook and loop fastener portion 644A coupled to the strap 642 and corresponding second hook and loop fastener portion 644B coupled with the bag 160 (FIG. 6J). The first and second hook and loop faster portions 644A, 644B may be coupled together to secure the bag 160 in the storage state (FIG. 6K).

FIG. 6L illustrates a fourth embodiment of a securement mechanism to maintain the bag 160 in the storage state (e.g., rolled up). The pull ring 134 of the cap 133 is configured to extend around the bag 160 in the storage state. In some embodiments, the pull ring 134 may be stretchable, to enable the pull ring 134 to extend around the bag 160 in the storage state.

FIGS. 6M-6T illustrate various embodiments of fold lines defining a collapsible structure for the bag 160. FIGS. 6M-6N illustrate a first embodiment of a number of fold lines 652 configured to transition the bag 160 from the usage state to the storage state. The fold lines 652 are organized into a number of transverse fold lines 652A and a number of longitudinal fold lines 652B, to enable the bag 160 to be fan folded, e.g., the sides of the bag 160 being folded inward along the longitudinal fold lines 652B and the bottom end 160B being folded towards the top end 160A along the transverse fold lines 652A, as illustrated in FIG. 6M. In some embodiments, the bag 160 may include a clasp 654 coupled to the bottom end 160B of the bag 160. In some embodiments, after the bag 160 is fan folded, the clasp 654 may be coupled with the catheter 101 to maintain the bag 160 in the storage state (FIG. 6N). In some embodiments, the transverse fold lines 652A may be spaced equidistant from each other.

FIGS. 60-6P illustrate a second embodiment of a number of fold lines 662 configured to transition the bag 160 from the usage state to the storage state. The fold lines 662 are organized into a number of transverse fold lines 662A. The transverse fold lines 662A enable the bag 160 to be fan folded (e.g., the bottom end 160B being folded towards the top end 160A along the fold lines 662). Thereafter, the sides of the bag 160 may be folded inward, such as in thirds, for example, as illustrated in FIG. 6P.

FIGS. 6Q-6R illustrate a third embodiment of a number of fold lines 672 configured to transition the bag 160 from the usage state to the storage state. The fold lines 672 are circumferential fold lines organized into an accordion or bellows engagement such that the bag 160 is collapsible along a longitudinal axis. The fold lines 672 are angled towards a longitudinal midpoint as illustrated in FIG. 6Q. In some embodiments, the bag 160 may include a handle 673, such as a pull loop, for example, coupled to the either the top end 160A or the bottom end 160B of the bag 160 to enable to the user to transition the bag 160 from the storage state to the usage state.

FIGS. 6S-6T illustrate a fourth embodiment of a number of fold lines 682 configured to transition the bag 160 from the usage state to the storage state. The fold lines 682 extend transversely across the bag 160 so that the bag 160 may be folded into the storage state. The bag 160 includes a number of holes 684 aligned longitudinally along a first side 660A and an opposite second side 660B. A thread 686 is threaded through the holes 684 along each of the sides 660A, 660B from the bottom end 160B to the top end 160A. The thread 868 further extends along each of the sides 660A, 660B from the top end 160A to the bottom end 160B. Each end of the thread 686 is then attached to the bag 160 at the bottom end 160B forming a thread loop 868A (FIG. 6T). During use, the user may pull on the thread loop 686A in a bottom direction to collapse the bag 160 along the fold lines 682 and transition the bag 160 from the usage state to the storage state.

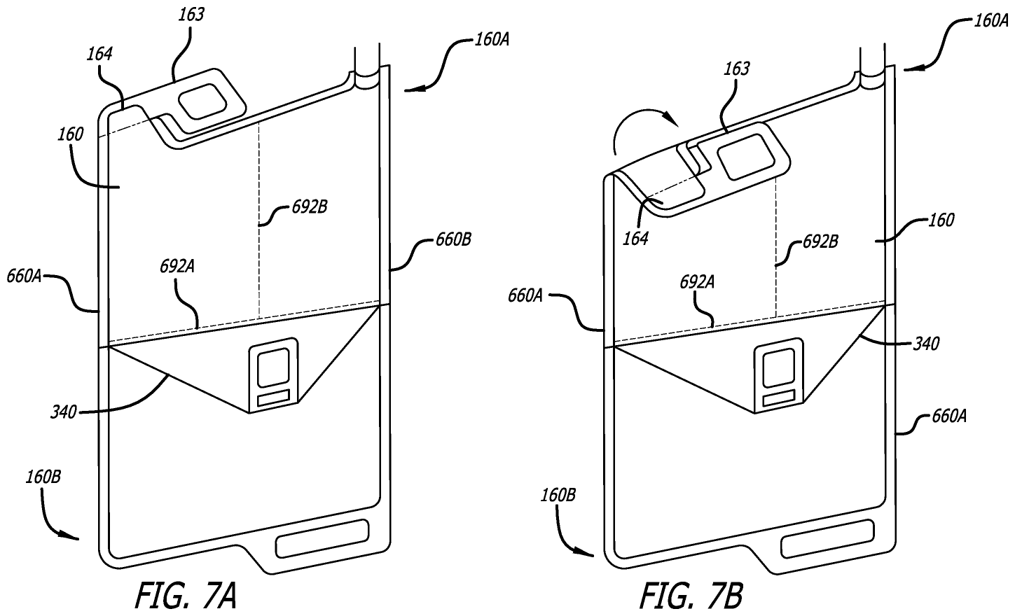

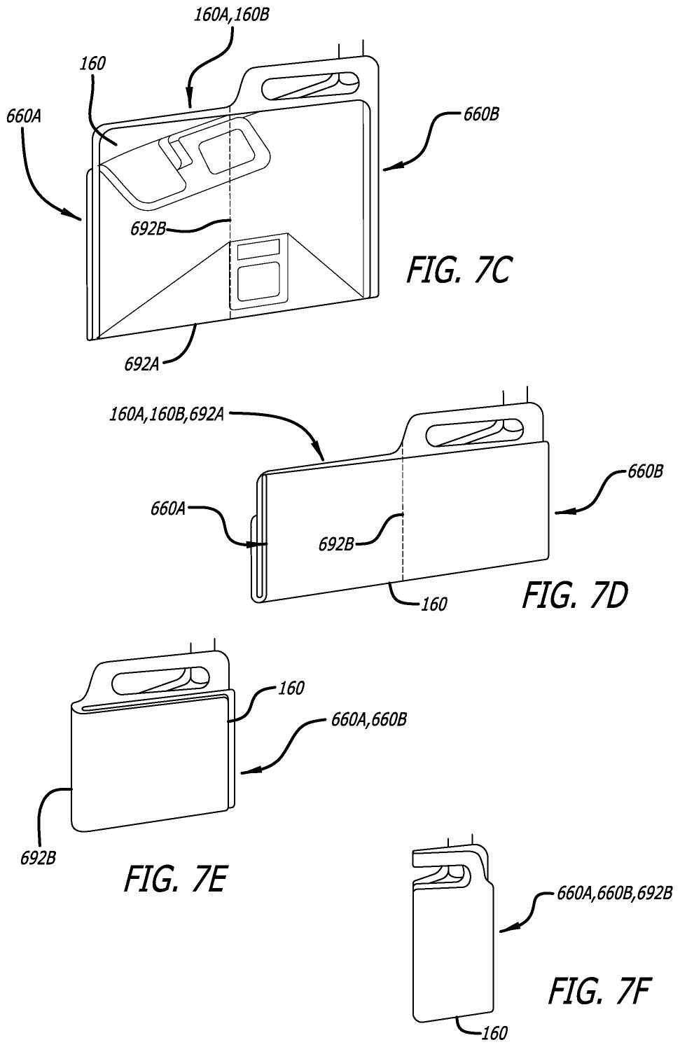

FIGS. 7A-7F illustrate perspective views of the bag 160 in sequential states of transitioning the bag 160 from the usage state to the storage state, in accordance with some embodiments. The bag 160 includes the storage bag 340 (FIG. 3). The storage bag 340 is detachably coupled to the bag 160, and the pull tab 163 is coupled to the frangible band 164, as illustrated in FIG. 7A. The bag 160 includes a transverse fold line 692A and a longitudinal fold line 692B. The frangible band 164 may be folded toward the bottom 160B of the bag 160 (FIG. 7B). The bag 160 is folded longitudinally upward a first time along the transverse fold line 692A so that the bottom end 160B is positioned adjacent the top end 160A (FIG. 7C). The bag 160 is longitudinally folded upward a second time so that the transverse fold line 692A is positioned adjacent the top end 160A and the bottom end 160B (FIG. 7D). The bag 160 is then folded transversely a first time along the longitudinal fold line 692B so that the first side 660A is positioned adjacent the second side 660B (FIG. 7E). The bag 160 is then folded transversely a second time so that the longitudinal fold line 692B is positioned adjacent the first side 660A and the second side 660B (FIG. 7F).

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A urinary catheter system, comprising:
a urinary catheter;
a urine collection bag defining a top end and a bottom end, the urine collection bag coupled with the urinary catheter via a hub at the top end, the urine collection bag comprising:
an inlet opening coupled with the hub, the inlet opening in fluid communication with a lumen of the urinary catheter via a lumen of the hub; and
an outlet configured to transition from a closed state to an opened state to allow urine to be drained from the urine collection bag; and
a storage bag separably attached to the urine collection bag, the storage bag configured to receive therein the urinary catheter and the urine collection bag in a storage state via an opening of the storage bag.

2. The system according to claim 1, wherein the urinary catheter is a male urinary catheter.

3. The system according to claim 1, further comprising a catheter cap having a pull ring, the catheter cap configured for attachment to a distal end of the urinary catheter both before and after use of the urinary catheter.

4. The system according to claim 1, wherein the urine collection bag is configured to be rolled up from the bottom end to the top end to transition the urine collection bag from the usage state to the storage state.

5. The system according to claim 1, wherein the urinary catheter is pre-attached to the urine collection bag.

6. The system according to claim 1, wherein the storage bag is separably attached to bottom end of the urine collection bag.

7. The system according to claim 1, wherein the storage bag is separably attached to the urine collection bag along a tear line.

8. The system according to claim 1, wherein the storage bag is separably attached to either one of a front panel or a back panel of the urine collection bag.

9. The system according to claim 1, wherein the storage bag includes a closure mechanism configured to securely contain the urine collection bag within the storage bag.

10. The system according to claim 9, wherein the closure mechanism includes a displaceable sealing member configured to define a fluid tight seal of storage bag opening when the displaceable sealing member is displaced across the storage bag opening.

11. The system according to claim 1, wherein the urine collection bag includes a handle disposed along a perimeter edge of the urine collection bag, the handle including a finger loop.

12. The system according to claim 11, wherein the handle extends away from perimeter edge.

13. The system according to claim 10, wherein the handle is disposed along a bottom perimeter edge of the urine collection bag.

14. The system according to claim 1, wherein the hub includes a one way valve disposed in line with the lumen of the hub, the one way valve configured to allow urine to flow into the urine collection bag via the lumen of the hub and prevent urine from flowing out of the urine collection bag via the lumen of the hub.

15. The system according to claim 1, wherein:
the hub includes:
a first hub portion coupled with the urine collection bag; and
a second hub portion coupled with the urinary catheter, and
the first and second hub portions define a continuity of the lumen of the hub when
the first and second hub portions are coupled together.

16. The system according to claim 15, wherein the second hub portion is detachably coupled with the first hub portion.

17. The system according to claim 15, wherein the second hub portion is coupled with the first hub portion via a hinge so that rotation of the second hub portion with respect to the first hub portion transitions the first and second hub portions between a coupled state and a decoupled state.

18. The system according to claim 15, wherein:
the first hub portion includes side port in fluid communication with the lumen of the hub, and
the first and second hub portions are slidably couple together so that the second hub portion is slidable between a depressed state with respect to the first hub portion and an extended state with respect to the first hub portion, such that:
when the second hub is in the extended state, the side port is in fluid communication with the lumen of the hub, thereby allowing the urine to flow out of the urine collection bag via the side port, and
when the second hub is in the depressed state, the side port is not in fluid communication with the lumen of the hub, thereby preventing the urine from flowing out of the urine collection bag via the side port.

19. The system according to claim 1, wherein the urine collection bag is longitudinally extendable between the storage state and the usage state.

20. The system according to claim 1, wherein the urine collection bag is longitudinally collapsed in the storage state.

21. The system according to claim 1, wherein the urine collection bag includes one or more straps configured to wrap around the urine collection bag in the storage state.

22. The system according to claim 21, wherein the one or more straps extend away from a perimeter edge of the urine collection bag.

23. The system according to claim 21, wherein:
each of the one or more straps includes one or more securing apertures extending through the respective strap, and
the one or more securing apertures are configured to threadably receive the respective strap therethrough to secure the respective strap.

24. The system according to either claim 21, wherein the one or more straps include an attachment device including a snap fastener or a hook and loop fastener.

25. The system according to claim 3, wherein the pull ring of the catheter cap is configured to stretch so as to enable to the pull ring to extend around the urine collection bag in the storage state.

26. The system according to claim 1, wherein:
the urine collection bag includes a rod extending along a bottom perimeter of the urine collection bag, and
the rod enables the user to roll up the urine collection bag from the bottom end to the top end.

27. The system according to claim 1, wherein:
the outlet includes a frangible band extending across a corner perimeter of the urine collection bag,
the frangible band includes pull tab configured to tear the frangible band when pulled by the user,
tearing the frangible band breaches a seal of the corner perimeter defining an outlet opening of the urine collection bag, and
the outlet opening allows for drainage of the urine from the urine collection bag.

28. The system according to claim 27, wherein the frangible band is tethered to the urine collection bag.

29. The system according to claim 1, wherein the outlet includes:
an outlet port defining an outlet port opening in fluid communication with an interior of the urine collection bag; and
an outlet cap sealably coupled with the outlet port, the outlet cap configured for separation from the outlet port to allow for drainage of the urine from the urine collection bag via the outlet port opening.

30. The system according to claim 29, wherein the outlet cap is hingedly coupled with the outlet port.

31. The system according to claim 29, wherein the cap is threadably coupled with the outlet port.

32. The system according to claim 1, wherein:
the outlet includes an outlet port defining an outlet port opening in fluid communication with an interior of the urine collection bag, and
the outlet port includes a frangible film extending across the outlet port opening, the frangible film defining a film seal of the outlet port opening.

33. The system according to claim 32, wherein the frangible film is a paper material configured to dissolve upon contact with the urine such that, during use, contact of the urine with the paper material breaches the film seal allowing for drainage of the urine from the urine collection bag via the outlet port opening.

34. The system according to claim 32, wherein the outlet port includes a piercing member tethered thereto, the piercing member configured to pierce the frangible film, thereby breaching the film seal to allow for drainage of the urine from the urine collection bag via the outlet port opening.

35. The system according to claim 34, wherein the piercing member includes a piercing member body defining a lumen of the piercing member, the piercing member configured to couple with the outlet port so that the lumen of the piercing member is in fluid communication with the outlet opening.

36. The system according to claim 1, wherein:
the outlet includes a first pull tab coupled with the front panel and a corresponding second pull tab coupled with the back panel,
each of the first and second pull tabs extend away from a corner perimeter of the urine collection bag
in use, pulling the first and second pull tabs apart from each other breaches a seal of the corner perimeter defining an outlet opening of the urine collection bag to allow for drainage of the urine from the urine collection bag via the outlet opening.

37. The system according to claim 36, wherein each of the first and second pull tabs includes a finger aperture.

38-70. (canceled)
